(19) United States
(12) Patent Application Publication
(10) Pub. No.: US 2026/0151326 A1
(43) Pub. Date: Jun. 4, 2026

(54) COMPOSITIONS COMPRISING EPERUA FALCATA EXTRACTS FOR SKIN TONE MANAGEMENT

(71) Applicant: L'OREAL, Paris (FR)

(72) Inventors: Rebecca BARRESI, Robbinsville, NJ (US); Ying CHEN, Montville, NJ (US); I-chien LIAO, Princeton, NJ (US); Jyotsna PATURI, Cupertino, CA (US); Maggie SU, Clark, NJ (US); Xiaoming HUANG, Shanghai (CN)

(73) Assignee: L'OREAL, Paris (FR)

(21) Appl. No.: 18/968,415

(22) Filed: Dec. 4, 2024

(51) Int. Cl. A61K 8/9789; A61Q 19/00

(52) U.S. Cl. CPC A61K 8/9789; A61Q 19/00

(57) ABSTRACT

Methods of treating skin by applying compositions that include extracts of Eperua falcata. The compositions are useful for skin tone management.

### FIELD OF THE DISCLOSURE

The present disclosure relates to compositions including extracts of Eperua falcata and their use for topical application to the skin for skin tone management. The Eperua falcata extracts and compositions comprising them are useful for improving the appearance of skin and, in particular, to manage skin tone and to prevent and treat pigmentation disorders.

### BACKGROUND

Skin is a unique and complex organ extending over the entire body. It is critical for protecting the body from external threats, controlling body temperature, and preventing loss of moisture. It also serves as a sensory organ and produces vitamin D. There are different types of skin at different locations of the body. For example, facial skin is different from that of the scalp, and even the skin on the palm of the hand is different than skin on the back of the hand.

Skin makes up the integumentary system, which includes three layers: the epidermis, the dermis, and the hypodermis. The epidermis is the outermost layer of skin and the first line of defense from external threats such as microbes. The human epidermis is principally composed of keratinocytes contains other types of cells including the melanocytes and the Langerhans' cells. Each of these cell types contribute, through their specific function, to the essential role played by the skin.

Skin, of course, comes in a variety of appearances, textures, hues, coloration and the like. Skin coloration is affected by various pigments including those in blood vessels in the deeper layers of the skin, as well as pigments that may be present throughout various layers. These pigments include carotenoids which can produce an orange-like tint to skin. Melanins, however, such as eumelanin, pheomelanin, are generally the predominant pigments in most human skin and are found in melanocytes located in the epidermis.

Variations in skin coloration or skin tone can be due to variations in the type and concentrations of pigments in the skin and on how many melanocytes a person has. While some variation of pigment in an individual's skin is normal, other pigment variations or unevenness can be an indication of a skin disorder.

Dyschromia is a general term indicating alteration of color of skin, hair or nails and may appear as skin tone unevenness. Darker portions or irregularities of skin tone are typically referred to as hyperpigmentation, whereas lighter portions or irregularities are typically referred to as hypopigmentation. The locus of these portions or irregularities are often referred to as “spots” or “patches.” These spots or patches can be caused by or associated with, for example, as acne scarring, atopic dermatitis lesions, psoriasis, or contact dermatitis; associated with mechanical stressors; associated with post inflammatory pigmentation; or associated with aging, genetic, sun exposure, or hormonal factors.

Overall, reducing or preventing excess pigment associated with particular areas of the skin represents a particular need state. There are existing treatments available for reducing or preventing excess pigment. For example, procedures such as laser or pulsed light treatments, and chemical peels are commonly used for this purpose. Topical treatments for reducing or preventing excess pigment are also known. Examples of active ingredients used for the purpose include, for example, azaleic acid and niacinamide. However, the present inventors have recognized the need for additional active ingredients, such as to provide improved efficacy, stability and/or formulation flexibility.

Furthermore, the formulation of environmentally-friendly cosmetic products, which are designed and developed considering environmental issues, is becoming a major goal in an effort to meet global challenges. It is therefore essential to propose more sustainable compositions, preparation processes and ingredients to address these environmental concerns.

In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

### SUMMARY OF THE DISCLOSURE

The instant disclosure relates to extracts of Eperua falcata and use of the extracts of Eperua falcata for topical application to the skin and, in particular, skin in need of skin tone management. The extracts of Eperua falcata, which may be incorporated into a pharmaceutical or cosmetic compositions, treat and prevent conditions that negatively impact skin tone. Moreover, application of the extracts of Eperua falcata to the skin, as shown in a skin model using 3-D reconstructed skin, surprisingly reduce the quantity of melanin or melanin density. It was also surprising to find that extracts of Eperua falcata influence pathways that relate to both melanin synthesis and melanin transport.

While extracts of Eperua falcata are known for use in skin care for anti-inflammatory purposes, the present inventors have found, as mentioned above, its usefulness related to skin tone and skin pigmentation management. Accordingly, the instant disclosure is drawn to the use of extracts of Eperua falcata in methods for treating the skin. In various embodiments, the extracts of Eperua falcata are applied to the skin in a pharmaceutical or cosmetic composition, which typically includes a physiologically acceptable carrier, for example, water and optionally water-soluble solvents. The pharmaceutical or cosmetic composition includes an amount of the extracts of Eperua falcata sufficient to ensure a therapeutically effective amount of the extracts of Eperua falcata is administered to the skin during use.

The extracts of Eperua falcata are useful for treating skin in need of skin tone management or pigmentation management. For example, the extracts of Eperua falcata surprisingly downregulate genes associated with melanin synthesis such as MC1R (a melanocytic Gₛ protein), DCT L (dopachrome tautomerase), and TYRP1 (tyrosinase-related protein 1). The inventors have also found that the extracts of Eperua falcata surprisingly downregulate genes associated with vesicle transport of melanin such as RAB27A.

Due to their depigmenting properties, including those described above, the extracts of Eperua falcata are useful to manage skin tone; provide/enhance skin tone evenness; reduce undesired pigmentation; reduce melanin density; improve the appearance of the skin; treats the skin, depigments the skin, lightens the skin and/or brightens the skin; and/or treats pigmentation disorders. The extracts of Eperua falcata may further be used for preventing or treating dyschromia, reducing, preventing or treating hyperpigmentation, reducing preventing or treating hyperpigmentation disorders, reducing, preventing or treating UV-induced pigmentation, or reducing, preventing or treating melasmic skin.

The extracts of Eperua falcata are often incorporated into cosmetic compositions for application to the skin. Pharmaceutical and cosmetic composition typically include extracts of Eperua falcata and one or more physiologically acceptable carrier, for example water. Nonlimiting examples of physiologically acceptable carriers include water, water soluble solvents such as alcohols, polyols, and glycols, fatty compound such as oils, triglycerides, fatty acids, fatty alcohols, and the like. Pharmaceutical and cosmetic compositions include lotions, creams, serums, sprays, emulsions, gels, powders, dispersions, ointments, sticks, pastes, and foams.

According to a first aspect of the invention, methods for treating skin comprising topically applying a composition comprising a therapeutically effective amount of one or more extracts of Eperua falcata to skin in need of skin tone management, such as hyperpigmented skin—is provided. The method may further include performing a procedure selected from laser therapy, microneedling, dermabrasion, radio frequency treatment, cryotherapy, chemical peel, or combinations thereof on the skin in need of skin tone management, before, after or during the topical application of the composition.

The skin in need of skin tone management may be skin associated with or having dermatological skin conditions such as one or more of: acne scarring, atopic dermatitis lesions, psoriasis, or contact dermatitis; hyperpigmentation from mechanical stressors; hyperpigmentation driven by hormonal factors such as melasma or chloasma; hyperpigmentation from sun or ultraviolet radiation exposure; hyperpigmentation driven by genetic and/or aging factors such as age spots, freckles, and lentigines; and combinations thereof. According to certain embodiments, the skin in need of skin tone management is, in particular, skin associated with hyperpigmentation from mechanical stressors; hyperpigmentation driven by hormonal factors such as melasma or chloasma; hyperpigmentation from sun or ultraviolet radiation exposure; hyperpigmentation driven by genetic and/or aging factors such as age spots, freckles, and lentigines; and combinations thereof. According to certain other embodiments, the skin in need of skin tone management is, in particular, skin having hyperpigmentation from mechanical stressors; hyperpigmentation driven by hormonal factors such as melasma or chloasma; hyperpigmentation driven by genetic and/or aging factors such as age spots, freckles, and lentigines; and combinations thereof. According to other embodiments, the skin in need of skin tone management is susceptible to the formation of enhanced pigmentation spots.

According to another aspect of the invention, a composition including extracts of Eperua falcata, a carob seed extract, and 2-oleamido-1,3-octadecanediol 2-oleamido-1,3-octadecanediol is provided. In certain embodiments, the one or more extracts of Eperua falcata are present in a concentration from about 0.001 to about 25% by weight and wherein the carob seed extract is present in a concentration from about 0.001 to about 30% by weight, and the 2-Oleamido-1,3-Octadecanediol is present in a concentration from about 0.0001 to about 5% by weight. The composition may be substantially free of sunscreen active agents or other pigmentation agents, or substantially free of both sunscreen active agents and other pigmentation agents. The composition may further include niacinamide and acetyl tripeptide-9.

### BRIEF DESCRIPTION OF THE DRAWINGS

Implementation of the present technology is described, by way of example only, with reference to the attached figures, wherein:

FIG. 1 shows the results of melanin quantification via Fontana-Masson staining, after treatment with compositions consistent with embodiments of the present invention and comparative compositions.

FIG. 2 shows images of reconstructed skin using multiphoton microscopy after treatment with compositions consistent with embodiments of the present invention and comparative composition.

FIG. 3 shows the results of melanin quantification via multiphoton microscopy, after treatment with compositions consistent with embodiments of the present invention and comparative composition.

FIG. 4 shows gene expression data gathered after firstly treating with either compositions consistent with embodiments of the present invention or comparative compositions, and secondly followed by exposure to deep UV radiation.

FIG. 5 shows a spider plot of various aspects of clinical efficacy of compositions consistent with embodiments of the present invention and comparative composition.

The various aspects of the disclosure are not limited to the results, arrangements, and representations shown in the drawings.

### DETAILED DESCRIPTION OF THE DISCLOSURE

The instant disclosure is drawn to compositions including extracts of Eperua falcata and their topical use for treating skin. Eperua falcata is a species of flowering plant in the family Fabaceae, and is native to northern South America. It is also known as Dimorpha falcata or the bootlace tree. Its timber is valued in construction uses such as in shingles and telephone poles.

Extracts of Eperua falcata include any of various extracts related to Eperua falcata. The term “Eperua falcata” in accordance with the present disclosure is any portion of the Eperua falcata plant, not limited to bark, flowers, roots, stems, leaves, seeds, and fruits. In certain notable embodiments, Eperua falcata upon which the extraction is performed includes at least the bark of the plant.

The extraction process may include any of various known extraction procedures including for example grinding or maceration, washing, soaking, filtration to removing solids, drying, and the like. Temperatures that are elevated (above ambient temperature) may be employed, as may be high pressure, microwave or ultrasonic treatments or other common conventional processes. The extraction may involve treating the plant with a solvent such as water or organic solvents. In certain notable embodiments the solvent includes or consists predominantly of water and the extract may be an aqueous extract.

According to certain notable embodiments, the extract of Eperua falcata may be provided in a vehicle that is liquid such as one including water or as a particulate, such as one including a carrier such as any of various materials such as starches or other carbohydates, proteins, or other carriers. In one embodiment the extract of Eperua falcata is an aqueous extract of the bark of Eperua falcata Aubl and is supplied in a maltodextrin carrier. One such example of a suitable extract of the bark of Eperua falcata Aubl is commercially supplied by BASF corporation of Ludwigshafen, Germany, under the name Eperuline.™

As described above, compositions useful for the invention described herein are useful for treating and preventing dyschromia and hyperpigmentation. Dyschromia is a general term indicating alteration of color of skin, hair or nails and may appear as skin tone unevenness. Darker portions or irregularities of skin tone are typically referred to as hyperpigmentation. The locus of these portions or irregularities are often referred to as “spots” or “patches.” Use of the term pigment or pigmentation as used herein refers to pigments found in human skin, such as melanin, including common variants of melanin such as eumelanin and pheomelanin.

Compositions useful in the present invention including extracts of Eperua falcata are useful for treating particular skin in need of skin tone management such as skin that includes (for example, spots or patches of) skin having dyschromia, including hyperpigmentation, including but not limited to: dyschromic skin, hyperpigmentated skin, browned skin; skin associated with dermatological skin conditions including acne scarring, atopic dermatitis lesions, psoriasis, or contact dermatitis; skin that includes hyperpigmentation from mechanical stressors such as burns, scrapes and related skin injuries; hyperpigmentation driven by hormonal factors such as melasmic skin (melasma) or cholasmic skin (chloasma); hyperpigmentation from sun or ultraviolet radiation (UV) exposure; hyperpigmentation driven by genetic and/or aging factors such as age spots, freckles, and lentigines; skin exhibiting visible pigmentation spots; and/or skin that is susceptible to the formation of pigmentation spots.

According to certain embodiments, compositions useful in the present invention including extracts of Eperua falcata are useful for treating the skin in need of skin tone management in skin associated with dermatological skin conditions selected from a group consisting of: acne scarring, atopic dermatitis lesions, psoriasis, or contact dermatitis; hyperpigmentation from mechanical stressors; hyperpigmentation driven by hormonal factors such as melasma or chloasma; hyperpigmentation from sun or ultraviolet radiation exposure; hyperpigmentation driven by genetic and/or aging factors such as age spots, freckles, and lentigines; and combinations thereof.

According to certain embodiments, compositions useful in the present invention including extracts of Eperua falcata are useful for skin associated with one or more of: hyperpigmentation from mechanical stressors; hyperpigmentation driven by hormonal factors such as melasma or chloasma; hyperpigmentation from sun or ultraviolet radiation exposure; hyperpigmentation driven by genetic and/or aging factors such as age spots, freckles, and lentigines; and combinations thereof.

According to certain other embodiments, the skin in need of skin tone management is skin associated with one or more of: hyperpigmentation from mechanical stressors; hyperpigmentation driven by hormonal factors such as melasma or chloasma; hyperpigmentation driven by genetic and/or aging factors such as age spots, freckles, and lentigines; and combinations thereof.

Accordingly, compositions useful in the present invention and including extracts of Eperua falcata can be used for various purposes, such as but not limited to managing skin tone; providing/enhancing skin tone evenness; reducing undesired pigmentation; reducing melanin density; improving the appearance of skin; treating, depigmenting, lightening and/or brightening skin; and/or treating pigmentation disorders of any of the particular need states described in the paragraph above. The compositions useful in the present invention may further be used for preventing or treating dyschromia, preventing or treating hyperpigmentation, preventing or treating hyperpigmentation disorders, preventing or treating UV-induced pigmentation, or preventing or treating melasmic skin.

This discovery is significant, because while extracts of Eperua falcata had been known to be applied to skin to fight chronic low-level inflammation to enhance skin barrier function, such functionality does not correspond or necessarily relate to efficacy related to depigmentation or skin tone management.

The skin in need of skin tone management may be skin that is susceptible to the formation of visible pigmentation spots. By visible pigmentation spots, it is meant such spots that are visible to eyes having normal human vision such as from about 12 inches away, such as from about 24 inches away).

The extracts of Eperua falcata are often incorporated into a pharmaceutical or cosmetic composition for application to the skin. Pharmaceutical and cosmetic compositions typically include one or more extracts of Eperua falcata and a physiologically acceptable carrier. A “physiological carrier” as used herein is a carrier that is appropriate and safe for application to the skin of a human. A particularly common physiologically acceptable carriers is water. However, physiologically acceptable carriers can be oil, fats, organic solvents, and the like, provided they are appropriate and safe for application to the skin. Nonlimiting examples of physiologically acceptable carriers include water, water-soluble solvents such as alcohols, polyols, and glycols, fatty compound such as oils, triglycerides, fatty acids, fatty alcohols, and the like. The pharmaceutical and cosmetic compositions of the instant disclosure can be lotions, creams, serums, sprays, emulsions, gels, powders, dispersions, ointments, sticks, pastes, or foams. Oil in water emulsions are particularly notable.

Extracts and compositions of the present invention are surprisingly capable of downregulating one or more genes associated with melanin synthesis. In particular, in a preferred embodiment, the one or more extracts of Eperua falcata downregulate one or more of: DCT L (L-dopachrome tautomerase), and TYRP1 (tyrosinase-related protein 1). DCT L, L-dopachrome tautomerase is an enzyme that plays a role in the melanin biosynthetic pathway by converting dopachrome to 5,6-dihydroxyindole-2-carboxylic acid (DHICA). DCT is part of the tyrosinase-related protein family, along with tyrosinase (Tyr) and Tyrp1, which are also melanogenic enzymes. TYRP 1 provides instructions for making an enzyme called tyrosinase-related protein 1. Tyrosinase-related protein 1 is involved in the production of melanin and melanosome biogenesis.

Furthermore, extracts and compositions of the present invention are surprisingly capable of downregulating one or more genes associated with melanin transport, such as RAB27A. Rab27a interacts with proteins from the MLPH and MYO5A genes to form a complex that moves melanosomes to the edges of melanocytes.

The therapeutically effective amount of the extracts of Eperua falcata will vary depending on the particular extract of Eperua falcata. Nonetheless, in various embodiments, the therapeutically effective amount may be from about 1 μg to about 50 mg (50,000 μg) per cm² of skin. In further embodiments, the therapeutically effective amount of the extract of Eperua falcata is from about 1 μg to about 40 mg, about 1 μg to about 30 mg, about 1 to about 20 mg, about 1 μg to about 10 mg, about 1 μg to about 8,000 μg, about 1 μg to about 5,000 μg, about 1 μg to about 2,000 μg, about 1 μg to about 1,000 μg, about 10 μg to about 40 mg, about 10 μg to about 30 mg, about 10 to about 20 mg, about 10 μg to about 10 mg, about 10 μg to about 8,000 μg, about 10 μg to about 5,000 μg, about 10 μg to about 2,000 μg, about 10 μg to about 1,000 μg, about 100 μg to about 50 mg, about 100 μg to about 40 mg, about 100 μg to about 30 mg, about 100 μg to about 20 mg, about 100 μg to about 10 mg, about 100 μg to about 8,000 μg, about 100 μg to about 5,000 μg, about 100 μg to about 2,000 μg, about 100 μg to about 1,000 μg, about 500 μg to about 50 mg, about 500 μg to about 40 mg, about 500 μg to about 30 mg, about 500 μg to about 30 mg, about 500 μg to about 20 mg, about 500 μg to about 10 mg, about 500 to about 8,000 μg, about 500 μg to about 5,000 μg, about 500 μg to about 2,000 μg, or about 500 μg to about 1,000 μg per cm² of skin.

The extracts of Eperua falcata are typically formulated with a physiologically acceptable carrier and applied to the skin as a pharmaceutical or cosmetic composition. The amount of the one or more extracts of Eperua falcata will vary depending on their activity, use, and interaction with other ingredients that may optionally be included in the pharmaceutical or cosmetic composition. Nonetheless, in various embodiments, the pharmaceutical or cosmetic composition include about 0.01 to about 10 wt. % of the one or more extracts of Eperua falcata, based on the total weight of the pharmaceutical or cosmetic composition.

In further embodiments, the pharmaceutical or cosmetic composition include about 0.01 to about 8 wt. %, about 0.01 to about 5 wt. %, about 0.01 to about 3 wt. %, about 0.01 to about 1 wt. %, about 0.01 to about 0.5 wt. %, about 0.05 to about 10 wt. %, about 0.05 to about 8 wt. %, about 0.05 to about 5 wt. %, about 0.05 to about 3 wt. %, about 0.05 to about 1 wt. %, about 0.075 to about 10 wt. %, about 0.075 to about 8 wt. %, about 0.075 to about 5 wt. %, about 0.075 to about 3 wt. %, about 0.075 to about 2 wt. %, about 0.075 to about 1 wt. %, of the extracts of Eperua falcata, on an active basis, excluding any carrier or vehicle (e.g., maltodextrin and the like), based on the total weight of the pharmaceutical or cosmetic composition.

Pharmaceutical and cosmetic compositions including the one or more extracts of Eperua falcata can be formulated in various forms, for example, lotions, creams, serums, sprays, emulsions, gels, powders, dispersions, ointments, sticks, pastes, and foams. In addition to a physiologically acceptable carrier, the pharmaceutical and cosmetic composition may optionally include one or more of the following:
Other biological active agents
Sunscreen active agents,
Emulsifying agents,
Emollients and humectants
Polymers, suspending agents and thickeners.
Additional solvents or carriers
Other ingredients

### Other Biological Benefit Agents

The pharmaceutical or cosmetic compositions of the instant disclosure may optionally include biological benefit agents other than the extract of Eperua falcata.

In one notable embodiment, the composition includes one or more of additional depigmentation agents as an additional benefit agent. The additional depigmentation agents may be selected from for example, niacinamide (NAM), certain ceramide or pseudo-ceramide compounds such as those having the structure R1-CHOH—CH(NH—COR2)(CH2OH) where R1 denotes a C11 to C21 alkyl radical, and R2 denotes a linear, C11-C19 hydrocarbon-based radical, and R2 is optionally substituted with a hydroxyl group in an alpha-position with respect to the carbonyl, and optionally comprises one or more ethylenic groups (an example of which is 2-OLEAMIDO-1,3-OCTADECANEDIOL also referred to herein as “OOD”); ectoin, resorcinol derivatives; 2-mercaptonicotinoyl glycine, hydroquinone; vitamin C and its derivatives, ferulic acid, tranexamic acid and derivatives thereof, gentisic acid, homogentisic, methyl gentisate or homogentisate, dioic acid, D pantheteine calcium sulphonate, lipoic acid, ellagic acid N-[(1,2-DIHYDRO-2-THIOXO-3-PYRIDINYL) CARBONYL]GLYCINE, water kiwi fruit (Actinidia chinensis) marketed by Gattefosse, an extract of Paeonia suffruticosa root, such as that sold by the company Ichimaru Pharcos under the name Botanpi Liquid B and kojic acid. According to certain embodiments, the compositions of the invention include at least one, two or all of niacinamide, ectoin and/or 2-oleamido-1,3-octadecanediol.

Other nonlimiting examples of biological benefit agents include skin barrier repair agents such as carob seed (Ceratonia siiqua) extracts, ceramides, hyaluronic acid. According to certain embodiments the skin barrier repair agent includes a carob seed (Ceratonia siiqua) extract such as GLYCO-REPAIR® available from Silab Cosmetic Ingredients.

Yet other nonlimiting examples of biological benefit agents include skin firming agents such as certain peptides such as caffeine, hyaluronic acid, retinoids, or acetyl tripeptide-9 the latter of which is available as Dermican SPB LS 9837 from BASF corporation.

Yet other biological benefit agents include walnut-derived peptides, ginger root extract, polysaccharides (including but not limited to galactomannans, pullulan, beta glucan, alpha glucan, Caesalpinia sphinosagum), oligosaccharides (including but not limited to oligo-galactomannans), wagonin, baicalin, ceramides/sphingolipids and its derivatives, resveratrol and its derivatives, niacinamide and its derivatives, transexamic acid (TXA), pomegranate extract, linoleic acid, linolenic acid, olicacid, vitamin C, vitamin E/tocopherols and its derivatives, polyphenols, flavanols, dihydroflavanols, ellagic acid, flavonoids, collagen and its derivatives, arnica montanaextract, carotenoids and its derivatives, triglycerides, soybean oil, oleuropeins and its derivatives, phospholipids, beta-carotene, micro-algae and algae extracts (including but not limited to Chlorella vulgaris extract, dunaliellasalina extract), pre/pro/post-biotics (including but not limited to Lactococcus ferment lysate, lactobacillus ferment), linseed extract, proanthocyanins, anthocyanins, hydrocyacetophenone, GHK-Cu peptide, tripeptide-1, copper peptide, GHK-Mn, Acetyl Tetrapeptides, Tripeptide 10 Citrulline, Palmitoyl Hexapeptide-12, Palmitoyl Tripeptide-1, Lipospondin, Gexapeptide11, PKEK, GEKG, SA1-11, Tripeptide-3, Pentapeptide-18, Pentapeptide-3, Acetyl Octapeptide-1,3, antimicrobial peptides (including but not limited to αand ß defensins, cathelicindin, dermcidin), NMF derived peptides (including but not limited to histidine dipeptides, Urocanic acid, pyrrolidone carboxylic acid), Pal-KTTKS and other acyl pentapeptides, KEK and PKEK peptides, Palmitoyl Tetrapeptide-7, Palmitoyl Pentapeptide-4, Tropaeolum Majus Extract, glycoproteins, hyaluronic acid and its derivatives, sh-Polypeptide-5, sh-Polypeptide-11, sh-Oligopeptide-2, sh-polypeptide-6, wheat germ oil, octacosanol, wild yam root extract, bakuchiol, borage oil, geranium oil, protium heptaphyllumresin, hamamelis virginiana extract, citrus medica limonumoil, glutathione, Co-enzyme Q10, disodium acetyl glucosamine phosphate, fermented red ginseng extract, dipeptide diaminobutyroylbenzylamide diacetate, angelica polymorpha sinensis root extract, sericin, superoxide dismutase.

The compositions of the inventions may include vitamins such as ascorbates (for example, vitamin C, vitamin C derivatives, ascorbic, stability, acid, ascorbyl glucoside, ascorbyl palmitate, magnesium ascorbyl phosphate, sodium ascorbyl phosphate, tetrahexadecyl scorbate, ascorbyl phosphate, aminopin-3-aminopinamine) vitamin B, derivatives of vitamin B, vitamin B1-vitamin B12 and their derivatives including niacinamide, vitamin K, derivatives of vitamin K, vitamin H, vitamin D, vitamin D3, derivatives of vitamin D, vitamin E, derivatives of vitamin E, and mixtures thereof. Vitamin compounds may be included as a substantially pure material or as an extract obtained by suitable physical and/or chemical isolation from natural (eg, plant) sources.

While the selections of additional biological active agents may vary, according to one embodiment the additional biological active agents include a carob seed (Ceratonia siiqua) extract. According to other embodiments the additional biological active agents include a compound having the structure R1-CHOH—CH(NH—COR2)(CH2OH) where R1 denotes a C11 to C21 alkyl radical, and R2 denotes a linear, C11-C19 hydrocarbon-based radical, and R2 is optionally substituted with a hydroxyl group in an alpha-position with respect to the carbonyl, and optionally comprises one or more ethylenic groups such as 2-Oleamido-1,3-Octadecanediol. According to other embodiments the additional biological active agents include a carob seed (Ceratonia siiqua) extract and 2-Oleamido-1,3-Octadecanediol. In certain other embodiments the additional biological active agents include at least one, two, three, four five or all or all of niacinamide, ectoin, 2-Oleamido-1,3-Octadecanediol, carob seed (Ceratonia siiqua) and acetyl tripeptide-9.

The total amount of the one or more other biological active agents include in the pharmaceutical or cosmetic compositions will vary but may be from about 0.0001% to about 25%, more preferably from about 0.001% to about 10%, more preferably from about 0.01% to about 5%, and more preferably from about 0.25% to about 4%, based on the total weight of the pharmaceutical or cosmetic compositions. In certain notable embodiments, the composition includes niacinamide.

### Sunscreen Active Agents

The pharmaceutical or cosmetic composition of the instant disclosure may optionally contain sunscreen active agents, which can also be referred to as a UV filtering agent or UV-filters. As used herein, “sunscreen active agents” include both sun reflectors and physical sunscreen agents. Suitable sunscreen active agents may be organic or inorganic. A wide variety of standard organic or inorganic sunscreen active agents are suitable for use in accordance with the present invention.

While in certain embodiments of the invention, sunscreen active agents may be included in compositions of the invention, in certain other notable embodiments it is desirable to have the compositions be substantially free, essentially free or entirely free of such ingredients. As such the compositions may be provide beneficial depigmentation of pigmentation prevention without the complexities otherwise that might be introduced by including sunscreen active agents (e.g., difficulty in formulation, possible skin or eye irritation, and the like).

The amount of the one or more sunscreen active agents in the pharmaceutical or cosmetic compositions, if included, will vary. Nonetheless, in certain embodiments, the pharmaceutical or cosmetic compositions include from about 0.1% to about 25%, more often from about 0.5% to about 10%, of sunscreen active agents, based on the total weight of the pharmaceutical or cosmetic compositions.

Nonlimiting examples of organic sunscreen active agents include those ingredients that are registered by regulatory agencies in either the United States (via the FDA) or in Europe (via the European Commission). Examples of sunscreen active agents include: ethylhexyl salicylate, butyl methoxybenzoylmethane, ethylhexylmethoxycinnamate, octocrylene, phenylbenzimidazole sulfonic acid, terephthalilide dicamphora sulfonic acid, benzophenone-4-benzophenone-3 camphor methylbenzylidene, benzimidazylate, anisotriazine, ethylhexyltriazone, diethylhexylbutamidotriazone, methylene-bis-benzotriazolyltetramethylbutylphenol, dromethrazole trisiloxane, and mixtures thereof.

Nonlimiting examples of inorganic sunscreen agents include those that are registered via the regulatory bodies described above-such as nanopigments (with an average primary particle size: generally, between 5 nm and 100 nm, preferably between 10 nm and 50 nm), or aggregates, whether or not coated with metal oxides, such as, for example, titanium oxide nanopigments (amorphous or crystalline in the form of rutile and/or anatase), iron, zinc, zirconium or cerium oxides, and mixtures thereof. Suitable coating agents for inorganic sunscreen agents include alumina and/or aluminum stearate and silicones.

### Emulsifying Agents

The pharmaceutical or cosmetic composition of the present invention may optionally include an effective amount an emulsifying agent, a humectant, and/or a skin conditioning agent.

Nonlimiting examples of emulsifying agents include surface active ingredients such as condensation products of alkylene oxides with fatty acids (ie alkylene oxide fatty acid esters), condensation products of alkylene oxides with 2 moles of fatty acids (ie fatty acid alkylene oxide diesters), condensation products alkylene oxides with fatty alcohols (ie fatty alcohol alkylene oxide esters), condensation products of alkylene oxides with both fatty acids and fatty alcohols and condensation products of glycerol with fatty acids (and optional shorter chain organic acids such as citric acid) Other nonionic surfactants suitable for use in this application include sugar esters and polyesters, alkoxylated sugar esters and polyesters, C1-C30 fatty acid esters C1-C30 fatty alcohols, alkoxylated C1-C30 ester derivatives C1-C30 fatty alcohol fatty acids, alkoxylated C1-C30 fatty alcohol esters, polyglyceryl C1-C30 fatty acid esters, C1-C30 polyol esters, C1-C30 polyol esters, alkyl phosphates, lecithin, and the like.

Other surface active ingredients such as surfactants that foam or may be irritating to the skin if left on without rinsing within a few minutes may be omitted from compositions useful in the present invention. Accordingly, the compositions may be substantially free of irritating surfactants and/or surfactants that foam significantly (such as certain anionic surfactants and/or amphoteric surfactants). Examples of such surfactants include one or more of fatty sulphates, sulfonates, isethionates, amphoacetates, sulfosuccinates, betaines, and sultaines. Other nonionic foaming surfactants such as glucosides may also be omitted from the composition.

The total concentration of emulsifiers that may be included in the composition include from about 0.1 to about 5% such as from about 2% to about 5% by weight.

### Emollients and Humectants

Nonlimiting examples of emollients include fatty acids, fatty acids fatty acid esters including for example the reaction products of fatty acids with C2-C26 alcohols, lipids, ceramides, cholesterol, cholesterol esters, beeswax, petrolatum, mineral oil, among others. The emollients generally remain on the skin surface or in the stratum corneum and act as a moisturizer or lubricant and reduce water loss. The total concentration of emollients that may be included in the composition include from about 0% to about 98%, such as from about 2% to about 30%, such as from about 3% to about 20% by weight, such as from about 5% to about 15% by weight.

Humectants include those materials that hold or retain water. Such materials include polyols such as glycols (including propylene glycol, butylene glycol, hexylene glycol), glycerin and urea derivatives. The total concentration of humectants that may be included in the composition include from about 0% to about 98% such as from about 2% to about 30% about 3% to about 20% by weight, such as from about 5% to about 15% by weight.

### Polymers, Suspending Agents and Thickeners

The pharmaceutical or cosmetic composition of the present invention may optionally include one or more polymers, suspending agents, and/or thickeners preferably in a concentration effective to suspend the water-insoluble material in a dispersed form in the compositions or to modify the viscosity of the composition. Such concentrations will vary. Nonetheless, in certain embodiments, the pharmaceutical and cosmetic composition includes from about 0.1% to about 10%, more preferably from about 0.25% to about 5.0% of the one or more suspending agents, such as from about 0.25% to about 2%, based on the total weight of the composition. Nonlimiting examples include vinyl polymers, such as cross-linked acrylic acid polymers, called carbomer, taurate polymers, cellulose derivatives and modified cellulose polymers such as methyl cellulose, ethyl cellulose, nitrocellulose, carboxymethyl cellulose, crystalline cellulose, cellulose powder, polyvinylpyrrolidone, polyvinyl alcohol, guar gum, hydroxypropyl guar gum, gum Arabic, galactan, locust bean gum, pectin, agar, starch (rice, corn, potato, wheat), algal colloids (algae extract), polymers such as dextran, maltodextrin, succinoglycan, pullulan, starch-based polymers such as carboxymethyl starch, methyl starch, alginic acid polymers such as sodium alginate, alginic acid propylene glycol esters, acrylate polymers such as sodium polyacrylate, polyacrylate, polyacrylamide, polyethyleneimine and inorganic minutes water soluble material such as bentonite, aluminum magnesium silicate, laponite, hectonite, and anhydrous silicic acid.

### Additional Solvents or Carriers

Any of a variety of additional solvents or carriers may be included in the composition. The solvents may be selected from, for example, water, C2-C5 monoalcohols, and combinations thereof. In certain embodiments, the compositions include from about 25% to about 90% of additional solvents, such as from about 30% to about 75%, such as from about 40% to about 70% by weight. In certain notable embodiments, the additional solvents or carriers includes or consists of water.

### Other Ingredients

Any of a variety of other cosmetic ingredients may be included in the composition, such as for example, chelating agents, preservation agents, antioxidants, colorants, pH adjusters, and the like. These additional ingredients may be present in a concentration from about 0% to about 50%, such as from about 0.1% to about 15%, such as from about 0.5% to about 10% by weight.

### Methods

Compositions described herein can be used for one or more of the following: to manage skin tone, to provide/enhance skin tone evenness; to reduce undesired pigmentation; to reduce melanin density; to improve the appearance of skin, to treat skin, to depigment skin, to lighten skin, to brighten skin; and/or to treat pigmentation disorders of any of the particular need states described herein. The extracts of Eperua falcata may further be used for preventing or treating dyschromia, preventing or treating hyperpigmentation, preventing or treating hyperpigmentation disorders, preventing or treating UV-induced pigmentation, and/or preventing or treating melasmic skin.

According to embodiments of the invention a composition including at least one extract of Eperua falcata is applied to particular skin such as skin in need of skin tone management. The skin may include (for example, spots or patches of) skin having dyschromia. The skin may include hyperpigmentation, such as but not limited to: dyschromic skin, hyperpigmentated skin, browned skin; skin associated with dermatological skin conditions including acne scarring, atopic dermatitis lesions, psoriasis, or contact dermatitis; skin that includes hyperpigmentation from mechanical stressors such as burns, scrapes and related skin injuries; hyperpigmentation driven by hormonal factors such as melasmic skin (melasma) or cholasmic skin (chloasma); hyperpigmentation from sun or ultraviolet radiation (UV) exposure; hyperpigmentation driven by genetic and/or aging factors such as age spots, freckles, and lentigines; skin exhibiting visible pigmentation spots; and/or skin that is susceptible to the formation of pigmentation spots.

According to certain methods, compositions described herein may be topically applied to the skin of the face, neck, arms, legs or other body parts once or periodically such as once per week, once per day or multiple times per day.

According to certain other methods, the composition is applied to the skin and allowed to remain on the skin (such as at least about 15 minutes, such as at least 30 minutes, such as at least 1 hour, such as at least about 2 hours) without rinsing. Such “leave on” products may be suitably formulated without irritating and/or foaming surfactants.

According to certain other methods, a procedure selected from laser therapy, microneedling, dermabrasion, radio frequency treatment, cryotherapy, chemical peel, or combinations thereof is also performed on the skin in need of skin tone management. The procedure may be performed before, after or during the course of treatment with the topical application of the composition.

The procedure may be one conducted in a professional's (e.g., dermatologist's) office or could be one using a suitable at home device, e.g., dermabrasion, light therapy, microneedle, and the like.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

### Example 1

### Depigmentation Effects of Extracts of Eperua falcata

Human pigmented epidermis was reconstructed. NHK and NHM (4:1) were seeded onto an insert with a bovine collagen matrix as dermal substitute. After an immersion culture for keratinocyte proliferation, cells were exposed to an air-liquid interface to sustain keratinocyte differentiation. Then, all the tissue samples were incubated at 37° C. with 5% CO2 and saturated humidity. For all the test compositions to be evaluated, first stock solutions in (DMSO) was prepared. The working solution was made by diluting the stock solution into the culture medium. In addition to DMSO control, two compositions including extracts of Eperua falcata were evaluated: (1) Eperua falcata Bark Extract (EP) at 0.001%, and (2) a combination of the following Eperua Falcata Bark Extract (EP) at 0.001%, and Carob Seed Extract at 0.00485%, and 2-Oleamido-1,3-Octadecanediol at 0.002% (EP+CS+OOD).

The skin models were treated by adding tested composition or DMSO control to the culture medium, starting from Day 9 and replaced with freshly prepared culture medium every day until Day 17 (D9˜D17, 6 applications). Every experimental condition is done in 6 replicates (¼ of each epidermis for DOPA; ¼ of each epidermis for MMP, ½ of each epidermis for Hematoxylin-Eosin (HE) and Fontana-Masson (FM). The quality of skin models after treatment is controlled by histological and the melanocyte morphology is observed after Hematoxylin-Eosin (HE) & DOPA Staining.

After treatment with test compositions, the cytotoxicity of treatment was estimated according to histological structure of reconstructed epidermis (images using HE staining). The HE stained samples revealed no significant cytotoxicity after the DMSO control or application of Eperua falcata bark extract (EP), or Eperua falcata bark extract (EP)+Carob seed extract (CS)+2-Oleamido-1,3-Octadecanediol (OOD) (“EP+CS+OOD”).

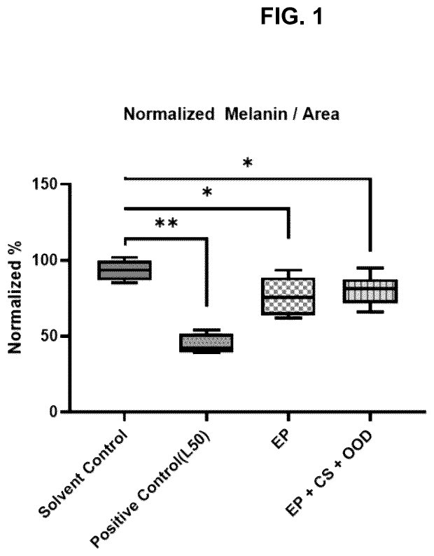

To quantify the amount of melanin present and thereby assess the depigmentation efficacy of the test compositions, the melanin present in skin model was stained on the histological slide using Fontana-Masson (FM) staining, then quantified by image analysis. Every epidermis slide is scanned using Nanozoomer®. For every epidermis, about 10˜15 images are extracted (optical microscopy, white light, magnification ×20). The area occupied by melanin is quantified with the help of Histolab® software. The results of the quantification of melanin (including mean and standard deviation) is shown in Table 1, below as well as in FIG. 1.

TABLE 1
Quantification of Melanin via Optical
Microscopy: Test Compositions

| | | Positive Control | | EP + CS + |
|---|---|---|---|---|
| | Solvent Control | (L50) | EP | OOD |
| | 98.88 | 43.89 | 65.91 | 94.62 |
| | 87.03 | 53.89 | 93.24 | 84.87 |
| | 85.1 | 39.07 | 84.3 | 73.35 |
| | 96.88 | 40.28 | 61.68 | 65.76 |
| | 101.9 | | 75.64 | 85.29 |
| | 89.78 | | | 77.32 |
| AVERAGE | 93.26 | 44.28 | 76.15 | 80.20 |
| STDEV | 6.88 | 6.72 | 12.97 | 10.19 |

It can be seen from the table and FIG. 1 that treatment with Eperua falcata bark extract alone (EP) or Eperua falcata bark extract in combination with carob seed extract and 2-Oleamido-1,3-Octadecanediol (EP+CS+OOD) reduces the amount of melanin quantity present in the skin samples. Note that in FIG. 1 (as well as in other figures included herein), “*” indicates a p value of 95% confidence, and “**” indicates a p-value of 99% confidence.

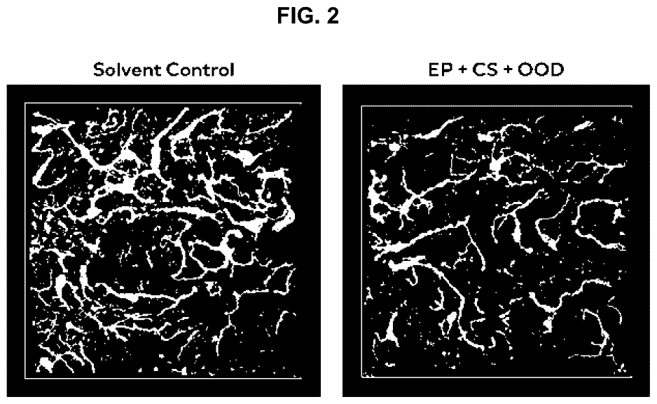

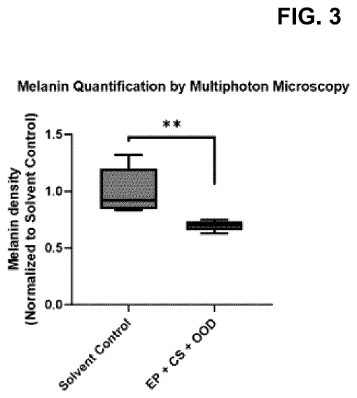

In order to provide another assessment of depigmentation efficacy, multiphoton imaging was performed with an up-right laser scanning microscope (Nikon A1RMP/FN1, Tokyo, Japan). 2PEF signals were excited by a femtosecond titanium-sapphire laser (MaiTai Deepsee, Spectra-Physics Santa Clara, California) adjusted to 760 nm, with typical 7 mW power at the sample. For high-resolution imaging, a high numerical-aperture, water immersion objective (25×, 1.1 NA, Nikon) was employed in all experiments. For melanin quantification, fast 2D XZ image acquisition was used: typically, around 50-100 images were recorded in every sample from basal layer to stratum cornem with dy=100 μm, one image corresponding to images of 1024×1024 pixels (dx=0.497 μm/pixel, dz=0.4101 μm/pixel). And 3D melanin image acquisition for visualization, we acquired typically one 300×300×100 μm3 XYZ image stack with 1 μm Z-step in every sample. One image corresponding to an image of 1024×1024 pixels (dx=dy=0.297 μm/pixel). Image processing were combined using Image J (W. Rasband, National Institutes of Health_), and 3-D image reconstructions with NIS Element (Nikon, Tokyo, Japan) for communication. Multiphoton images are shown in FIG. 2. Quantification of melanin (including mean and standard Deviation) is shown in Table 2 below as well as in FIG. 3.

TABLE 2
Quantification of Melanin via Multiphoton
Microscopy: Test Compositions

| | Solvent | EP + CS + |
|---|---|---|
| | Control | OOD |
| | 1.317801316 | 0.629583503 |
| | 1.161993742 | 0.728908075 |
| | 0.937297168 | 0.688444881 |
| | 0.84103659 | 0.667247281 |
| | 0.907360033 | 0.711077172 |
| | 0.834511151 | 0.749423156 |
| Average | 1.00 | 0.696 |
| STDEV | 0.196 | 0.043 |

It can again be seen from the table and figure that treatment with Eperua falcata bark extract in combination with carob seed extract and 2-Oleamido-1,3-Octadecanediol (EP+CS+OOD) reduces the amount of melanin present in the skin samples.

### Example 2

### Anti-Pigmentation Effects of Extracts of Eperua falcata

Human pigmented epidermis was reconstructed. NHK and NHM (4:1) were seeded onto an insert with a bovine collagen matrix as dermal substitute. After an immersion culture for keratinocyte proliferation, cells were exposed to an air-liquid interface to sustain keratinocyte differentiation. Then, all the tissue samples were incubated at 37° C. with 5% CO2 and saturated humidity. For all the test compositions to be evaluated, first stock solutions in (DMSO) was prepared. The working solution was made by diluting the stock solution into the culture medium. In addition to (1) DMSO control, two compositions including extracts of Eperua falcata were evaluated: (2) Eperua Falcata Bark Extract (EP) at 0.001% in DMSO and (3) the six-way combination of Eperua Falcata Bark Extract (EP) at 0.001%+Carob Seed Extract (CS) at 0.0485%+2-Oleamido-1,3-Octadecanediol (OOD) at 0.002%, +Acetyl Tetrapeptide-9 (AT9) at 0.0000023%+Niacinamide (NAM) at 0.02%+ectoin (ECT) at 0.002% in DMSO (“EP+CS+OOD+AT9+NAM+ECT”).

After treatment with test compositions, deep ultraviolet radiation (DUVR) exposure was delivered using a 1600 W Xenon arc solar simulator (Newport SOL-UV-6, serial number 1003) equipped with a Daily UV filter. During UV exposure, skin model samples were transferred to new12-well plates containing fresh DPBS. Exposure with 7.5 J/cm² Daily UV, Then the skin model samples were replaced with fresh medium content tested RMs or Solvent Control in 5% CO2 incubator. The exposure is starting from Day 9 and 4 times repeated exposure from Day 9 to Day 14. Samples were harvested at Day 15 for analysis. Every experimental condition is done in 6 replicates. The quality of skin models after treatment was controlled by histological and the melanocyte morphology is observed after Hematoxylin-Eosin & DOPA Staining. Note that no DUVR (and no test composition) was delivered to one sample as an untreated control. Another sample was treated with DMSO (solvent) only, followed by DUVR, as a solvent control.

After treatment with test compositions, the cytotoxicity of treatment was estimated according to histological structure of reconstructed epidermis (images using HE staining). The HE stained samples revealed no significant cytotoxicity after the DMSO control or application of Eperua falcata bark extract (“EP”), or Eperua falcata bark extract (EP)+Carob seed extract+2-Oleamido-1,3-Octadecanediol (OOD)+Acetyl Tetrapeptide-9 (AT9)+Niacinamide (NAM)+ectoin (ECT) (“EP+CS+OOD+AT9+NAM+ECT”).

Gene expression (RNA) analysis was conducted using QuantiGene RNA Multiplex assay (QGP, Affymetrix, Santa Clara, CA, U.S.A.). Gene expression evaluation epidermis separated from BPER in each reconstructed skin was conducted according to the manufacture's specifications. Gene expression modulation fold (MF) was calculated using the formula below, with MFI indicating Median Fluorescence Intensity, and HKG indicating Housekeeping genes.

$$Fold Change = [ ( MFI - Blank ) gene of interest / ( MFI - Blank ) HKG ] UV + RMs applied sample / [ ( MFI - Blank ) gene of interest / ( MFI - Blank ) HKG ] UV applied sample$$

The expression of all genes was initially analysed with QGP assay. Genes with a low signal or exceeding the detection limit (MFI<limit of detection or MFI>2000) or exhibiting a poor linearity in a gradient dilution test were reanalysed using RT-qPCR method. RNA was extracted from skin samples using MagMAX Kit (Ambion, Waltham, MA, U.S.A.) and the total RNA was used for first-strand cDNA synthesis. Quantitative PCR was performed using the Taqman DNA probe Mix and the Taqman gene expression Master kit (Invitrogen, Carlsbad, CA, U.S.A.). Relative quantification was performed using the 2\hat{ }-(ΔΔCt) method. GAPDH was used as HKG for RNA analysis.

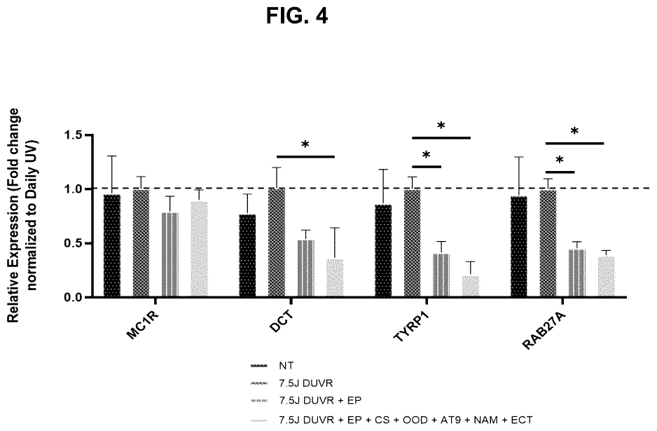

Key genes related to melanogenesis (MC1R, DCT, TYRP1) and melanin transfer process (RAB27A) were analyzed. The quantified gene expression data is depicted in Table 3 below. For clarity, no DUVR (and no test composition) was delivered to the sample labeled, “Untreated Control.” The sample labeled “DUVR (Solvent Control)” was treated with DMSO only, followed by DUVR. The same results are also shown in FIG. 4. In FIG. 4, the DUVR treated samples are labeled “7.5 DUVR.” For example, the sample treated with DMSO only, followed by DUVR is labeled “7.5 J DUVR.” The sample treated with ER before DUVT is labeled “7.5 J DUVR+EP,” and the sample treated with EP+CR+OOD+AT9+NAM+ECT before DUVT is labeled “7.5 J DUVR+EP+CR+OOD+AT9+NAM+ECT” The untreated control is labeled “NT.”

Gene expression following DUVR exposure reveals shifts to these key genes. It is demonstrated that Eperua falcata bark extract (EP) alone significantly decreases TYRP1 and RAB27A expression. TYRP1, the gene that codes for the enzyme tyrosinase-related protein 1, plays an important role in melanin synthesis and tyrosinase stabilization during the early melanogenesis process. Meanwhile, RAB27A is integral in the transport of synthesized melanin from melanocytes to its surrounding keratinocytes. It has been demonstrated that the expression of RAB27A mRNA and protein levels is positively correlated with melanin content in melanocytes. Therefore, it is reasonable to conclude that eperuline reduces melanin content by potentially targeting both early melanogenesis processes and reducing melanin transfer.

TABLE 3
Quantified Gene Expression following Deep UV Radiation

| | Untreated | DUVR (Solvent | | EP + CR + OOD + |
|---|---|---|---|---|
| | Control | Control) | EP | AT9 + NAM + ECT |
| MC1R | 1.31 | 1.13 | 0.87 | 0.92 |
| | 0.95 | 0.96 | 0.88 | 0.98 |
| | 0.62 | 0.92 | 0.63 | 0.80 |
| Average | 0.96 | 1.00 | 0.79 | 0.90 |
| Stdev | 0.35 | 0.11 | 0.14 | 0.09 |
| DCT | 0.83 | 0.89 | 0.47 | 0.45 |
| | 0.92 | 0.91 | 0.63 | 0.59 |
| | 0.58 | 1.23 | 0.52 | 0.06 |
| Average | 0.78 | 1.01 | 0.54 | 0.37 |
| Stdev | 0.18 | 0.19 | 0.08 | 0.27 |
| TYRP1 | 1.21 | 0.95 | 0.53 | 0.30 |
| | 0.80 | 0.93 | 0.38 | 0.26 |
| | 0.59 | 1.13 | 0.33 | 0.08 |
| Average | 0.87 | 1.00 | 0.41 | 0.21 |
| Stdev | 0.32 | 0.11 | 0.10 | 0.12 |
| RAB27A | 1.09 | 0.94 | 0.52 | 0.44 |
| | 1.20 | 0.95 | 0.44 | 0.36 |
| | 0.54 | 1.11 | 0.40 | 0.37 |
| Average | 0.94 | 1.00 | 0.45 | 0.39 |
| Stdev | 0.35 | 0.10 | 0.06 | 0.04 |

### Example 3

### Clinical Efficacy of Extracts of Eperua falcata for Skin Tone Management

A monocenter, double blinded, randomized split-face non-ablative fractional laser clinical study was conducted. A test composition (containing an extract of Eperua falcata (EP), carob seed extract (CS), 2-Oleamido-1,3-Octadecanediol (OOD), Acetyl Tetrapeptide-9 (AT9), niacinamide (NAM) and ectoin (ECT). This combination referred to as “EP+CS+OOD+AT9+NAM+ECT” was compared to AQUAPHOR, a medical OTC benchmark (“benchmark”) for barrier rejuvenation and anti-aging related endpoints. The study concluded with n=35 subjects (Fitzpatrick Type II [n=6] and Fitzpatrick Type III [n=29], age 57±5 years old). Following laser treatment, the test product was applied to the corresponding side two times daily for 28 days. Anti-aging endpoints (VISIA, OCT, Antera 3d imaging and clinical assessment of fine lines/wrinkles, dyschromia, skin tone evenness, skin radiance and brightness, overall healthy appearance, skin roughness-visual and tactile, and skin firmness-tactile) were measured at day twenty-eight (“D28”).

The test composition used had the following ingredients, shown in Table 4, below (apparent errors in total concentration are due to rounding).

TABLE 4
Example Facial Composition

| INGREDIENT | WT. % |
|---|---|
| Water | 73 |
| Polyols | 10 |
| Emollients | 2 |
| Emulsifiers | 4 |
| Additional Biological Actives | 0.1 |
| Polymers & suspending agents | 2.0 |
| 2-OLEAMIDO-1,3-OCTADECANEDIOL | 0.2 |
| ECTOIN | 0.2 |
| CERATONIA SILIQUA (CAROB) SEED | 0.5 |
| EXTRACT/CERATONIA | |
| SILIQUA SEED EXTRACT | |
| ACETYL TETRAPEPTIDE-9 | 0.003 |
| NIACINAMIDE | 2 |
| EPERUA FALCATA BARK EXTRACT | 0.1 |
| Other ingredients | 1 |

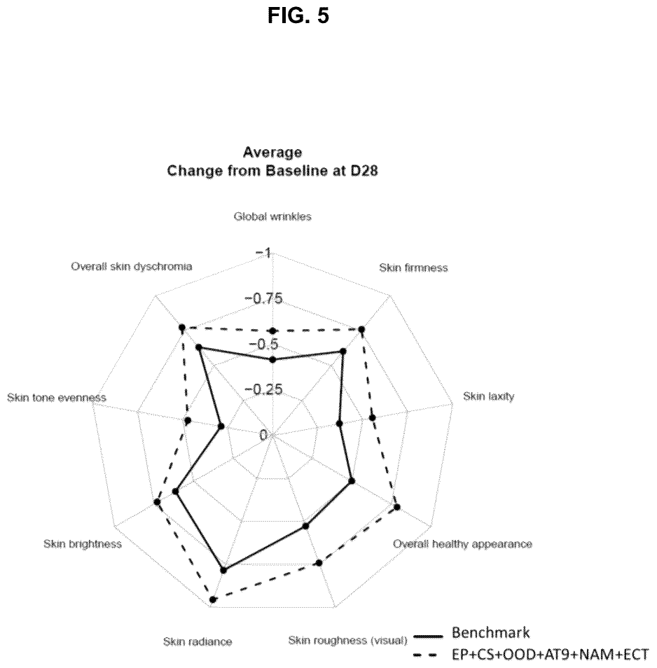

Clinical grading revealed significant improvement compared to the OTC benchmark on key skin tone management endpoints-skin tone evenness, skin brightness, skin radiance, and overall skin dyschromia are shown in the “spider plot” of FIG. 5. Each of these parameters also demonstrated clinically relevant changes (grading over −0.5).

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both an emollient and a nonionic surfactant. If a particular composition includes both an emollient and a nonionic surfactant, a single compound will serve as only the emollient or only as the nonionic surfactant (the single compound does not simultaneously serve as both the emollient and nonionic surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” (or “omitted” ingredients) as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure. According to certain notable embodiments, the cosmetic or pharmaceutical composition is substantially free of sunscreen active agents.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for treating skin comprising topically applying a composition comprising a therapeutically effective amount of one or more extracts of Eperua falcata to skin in need of skin tone management.

2. The method of claim 1 further comprising performing a procedure selected from laser therapy, microneedling, dermabrasion, radio frequency treatment, cryotherapy, chemical peel, or combinations thereof on the skin in need of skin tone management, wherein said performance of said procedure is before, after or during said topical application of the composition.

3. The method of claim 1, wherein the skin in need of skin tone management is hyperpigmented skin.

4. The method of claim 1, wherein the skin in need of skin tone management is skin associated with dermatological skin conditions selected from a group consisting of: acne scarring, atopic dermatitis lesions, psoriasis, or contact dermatitis; hyperpigmentation from mechanical stressors; hyperpigmentation driven by hormonal factors such as melasma or chloasma; hyperpigmentation from sun or ultraviolet radiation exposure; hyperpigmentation driven by genetic and/or aging factors such as age spots, freckles, and lentigines; and combinations thereof.

5. The method of claim 1, wherein the skin in need of skin tone management is skin associated with dermatological skin conditions selected from a group consisting of: hyperpigmentation from mechanical stressors; hyperpigmentation driven by hormonal factors such as melasma or chloasma; hyperpigmentation from sun or ultraviolet radiation exposure; hyperpigmentation driven by genetic and/or aging factors such as age spots, freckles, and lentigines; and combinations thereof.

6. The method of claim 1, wherein the skin in need of skin tone management is skin associated with dermatological skin conditions selected from a group consisting of: hyperpigmentation from mechanical stressors; hyperpigmentation driven by hormonal factors such as melasma or chloasma; hyperpigmentation driven by genetic and/or aging factors such as age spots, freckles, and lentigines; and combinations thereof.

7. The method of claim 1, wherein the skin in need of skin tone management is susceptible to the formation of enhanced pigmentation spots.

8. The method of claim 1, wherein the method improves the appearance of skin, reduces melanin density, depigments, lightens and/or brightens the skin.

9. The method of claim 1, wherein the composition comprising a therapeutically effective amount of one or more extracts of Eperua falcata reduces or prevents or reduces pigmentation.

10. The method of claim 1, wherein the composition is substantially free of sunscreen active agents.

11. The method of claim 1, wherein the composition further comprises a carob seed extract.

12. The method of claim 1, wherein the composition further comprises a compound having the structure R1-CHOH—CH(NH—COR2)(CH2OH) where R1 denotes a C11 to C21 alkyl radical, and R2 denotes a linear, C11-C19 hydrocarbon-based radical, and R2 is optionally substituted with a hydroxyl group in an alpha-position with respect to the carbonyl, and optionally comprises one or more ethylenic groups.

13. The method of claim 1, wherein the composition further comprises a carob seed extract and 2-oleamido-1,3-octadecanediol 2-oleamido-1,3-octadecanediol.

14. The method of claim 13, wherein the one or more extracts of Eperua falcata are present in a concentration from about 0.001 to about 25% by weight and wherein the carob seed extract is present in a concentration from about 0.001 to about 30% by weight, and the 2-Oleamido-1,3-Octadecanediol is present in a concentration from about 0.0001 to about 5% by weight.

15. The method of claim 13, wherein the composition further comprises niacinamide and acetyl tripeptide-9.

16. The method of claim 1, wherein the composition is substantially free of other pigmentation agents.

17. The method of claim 1, wherein the composition is allowed to remain on the skin without rinsing.

18. A composition for managing skin tone, wherein the composition comprises
a therapeutically effective amount of one or more extracts of Eperua falcata;
a carob seed extract; and
2-Oleamido-1,3-Octadecanediol.
